(19) United States
(12) Patent Application Publication
(10) Pub. No.: US 2012/0165227 A1
(43) Pub. Date: Jun. 28, 2012

(54) COMPOUNDS AND METHODS FOR DETECTION AND QUANTIFICATION OF CARBOXYLIC ACIDS

(75) Inventors: Liang Li, Edmonton (CA); Kun (Kevin) Guo, Edmonton (CA)

(73) Assignee: The Governors of the University of Alberta, Edmonton, AB (CA)

(21) Appl. No.: 13/382,303

(22) PCT Filed: Jul. 9, 2010
(86) PCT No.: PCT/CA2010/001064

§ 371 Date: Mar. 8, 2012

Related U.S. Application Data
(60) Provisional application No. 61/224,500, filed on Jul. 10, 2009.

(51) Int. Cl. G01N 33/50; G01N 27/62; C40B 40/04; C07C 211/48; C07C 67/00

(52) U.S. Cl. 506/15; 564/442; 560/1; 436/129

(57) ABSTRACT

The present disclosure includes compounds of Formula I: wherein R¹-R³, Ar, and n are as defined herein, and methods for the quantification of carboxylic acids in samples, specifically biological samples, using the compounds of Formula I. The compounds of Formula I are novel stable isotopic reagents that are useful in differential isotopic labeling methods.

### RELATED APPLICATIONS

This application is a PCT application that claims the benefit of priority of co-pending U.S. Provisional Patent Application 61/224,500 filed Jul. 9, 2010, which is herein incorporated in its entirety by reference.

### FIELD OF THE DISCLOSURE

The present disclosure is in the field of analysis of carboxylic acids in samples, in particular, using differential isotope labeling coupled with mass spectrometry.

### BACKGROUND OF THE DISCLOSURE

One of the early reports of using differential isotope labeling (DIL) for metabolite analysis was the use of the iTRAQ reagent, commonly known as the labeling reagent for peptides for quantitative proteomics, to label amino acids for quantitative analysis of these small molecules in urine and blood samples.¹ Fukusaki et al reported the use of ¹³C- and ¹²C-methylation to introduce differential isotope tags to flavonoids for relative quantification.² Yang et al described a LC/MS method for amino acid analysis involving derivatization with an N-hydroxysuccinimide ester of N-alkylnicotinic acid where the alkyl chain can contain deuterium, instead of hydrogen, to provide a differential isotope tag.³ Shortreed, et al reported the use of heavy and light isotopic forms of methyl acetimidate for the relative metabolome quantifications of amine-containing species.⁴ Guo et al used the reductive amination reaction to label amine-containing metabolites with ¹³C- and ¹²C-formaldehyde for relative metabolome quantifications.⁵ Ji et al reported the use of acetaldehyde d(4) to label and quantify the monoamine neurotranmitters in rat brain microdialysates.⁶ Abello et al developed isotope tagged pentafluorophenyl-activated esters of poly(ethylene glycol) to label amine-containing metabolites with multiplexing capability.^{7 13}C₄ labeled succinic anhydride and deuterated (D9) butanol have been used for labeling metabolites for relative metabolome analysis.⁸ While LC/MS is commonly used for detecting the differential isotope labeled metabolites, GC/MS has also been combined with chemical derivatization with isotope-coded reagents for metabolome analysis.⁹ It should be noted that a related method using isotope enriched media for cell culturing has been used for quantitative metabolomics.¹⁰⁻¹⁶ More recently, Guo and Li reported the use of dansyl chloride as a labeling reagent for analyzing amine- and phenol-containing metabolites.¹⁷

Phenacyl bromide has been used to form derivatives of carboxylic acids for analytical applications such as improving the performance of HPLC and UV detection.^{18,19} The synthesis of an isotope form of phenacyl bromide (5 hydrogen atoms in the benzene ring were replaced by 5 deuterium atoms) has been reported.²⁰ This reagent was used to label peptides for MS analysis.^{20,21}

### SUMMARY OF THE DISCLOSURE

Novel stable-isotope forms of p-dimethylaminophenacyl bromide (DmPA) have been prepared and shown to be useful as reagents for binary and multiplex quantification of carboxylic acids.

Accordingly, the present disclosure includes a compound of Formula I:

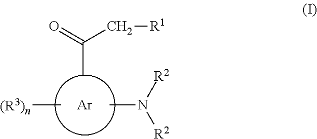

wherein
Ar is phenyl or naphthyl;
R¹ is a suitable leaving group;
R² is C₁₋₁₀alkyl in which one, two or three of the carbon atoms, with the exception of the carbon atom attached to the nitrogen, is optionally replaced with O and/or NR⁴ and one or more of the carbon atoms in R² is present as a carbon-13 isotope in amounts greater than the natural abundance of carbon-13 and/or one or more of the oxygen atoms, if present, in R² is present as an oxygen-18 isotope in amounts greater than the natural abundance of oxygen-18;
R³ is selected from C₁₋₆alkoxy and N(C₁₋₆alkyl)₂;
R⁴ is selected from H and C₁₋₆alkyl; and
n is 0, 1, 2, 3 or 4, and
salts and solvates thereof.

The present application also includes carboxylic acids derivatized with a compound of Formula I as defined above. It will be appreciated by those skilled in the art that any compound comprising at least one carboxylic acid moiety is capable of reacting with the compounds of Formula I to form the corresponding ester. It is an embodiment of the disclosure that the carboxylic acid is a metabolite found in a biological sample.

Also included in the present disclosure in a method of preparing an ester of a carboxylic acid comprising reacting a compound comprising at least one carboxylic acid with a compound of Formula I as defined above in the presence of a suitable base under conditions to form the ester of the one or more carboxylic acids.

The present application also includes a library comprising, consisting essentially of or consisting of two or more esters of a carboxylic acid, wherein the esters are formed by the reaction of a compound of Formula I as defined above with the carboxylic acid in the presence of a base.

The disclosure also includes a binary method of quantifying one or more carboxylic acids in first and second samples comprising:
(a) reacting an aliquot of a first sample with a compound of Formula I as defined above in the presence of a suitable base under conditions to form a first reaction mixture comprising a first ester of the one or more carboxylic acids;
(b) reacting an aliquot of a second sample with a compound having the same structure as the compound of Formula I, with the exception that R² contains an amount of carbon-13 and oxygen-18, if present, that corresponds to their natural abundance, in the presence of a suitable base under conditions to form a second reaction mixture comprising a second ester of the one or more carboxylic acids;
(c) combining the first and second reaction mixtures; and
(d) subjecting the combination of (c) to mass spectrometry analysis and quantifying an amount of carboxylic acids in the first sample relative to the second sample.

In an embodiment of the disclosure, one of the first or second sample comprises one or more standard carboxylic acids with known concentrations and the method provides an absolute quantification of the one or more carboxylic acids in the other of the first or second sample.

The present disclosure also includes a multiplex method of quantifying one or more carboxylic acids in three or more samples comprising:
(a) reacting aliquots of two or more samples, separately, each with a different compound of Formula I as defined above having a different isotope mass, in the presence of a suitable base under conditions to form two or more reaction mixtures each comprising a different ester of the one or more carboxylic acids;
(b) reacting an aliquot of a standard sample with a compound having the same structure as the compound of Formula I, with the exception that R² contains an amount of carbon-13 and oxygen-18, if present, that corresponds to their natural abundance, in the presence of a suitable base under conditions to form a standard reaction mixture comprising a standard ester of the one or more carboxylic acids;
(c) combining, in any order and combination, the reaction mixtures of (a) and (b); and
(d) subjecting the combination of (c) to mass spectrometry analysis and quantifying an amount of carboxylic acids in the two or more samples relative to the standard sample.

In an embodiment of the disclosure, where the standard sample comprises one or more standard carboxylic acids with known concentrations, the method provides an absolute quantification of the one or more carboxylic acids in the two or more samples.

Advantages of the compounds of Formula I as differential isotope labeling reagents, include, for example,
(i) the isotope reagents are readily synthesized and purified and are stable;
(ii) the acid reaction with the compounds of Formula I as defined above is simple, requiring no special equipment, and is fast (˜10-20 minutes). This reaction is broadly applicable to a wide range of carboxylic acids and produces little or no side reaction products;
(iii) the ¹²C/¹³C-Formula I-labeled metabolites do not show an isotope effect in the reversed phase LC (RPLC) separation. The differential isotope ion pairs are co-eluted and detected by MS and thus are subjected to the same degrees of matrix and/or ion suppression effect, which leads to high precision and accuracy for quantitative metabolite analysis;
(iv) by containing a dialkylamino group, the compound can be readily charged during the electrospray ionization (ESI) mass spectrometry (MS) process, thereby increasing the MS detection sensitivity greatly;
(v) the addition of the group corresponding to a compound of Formula I, minus the leaving group effectively increases the mass window for detection of the metabolite to at least 163 Da above the metabolite's original molecular weight, avoiding any signal interference raised from low-mass background molecules and contaminants commonly present in the ESI process;
(vi) Formula I derivatives of metabolites with known structures can form a library of standards from which absolute concentrations of these metabolites can be determined in any biological sample and metabolite identification can be done based on accurate mass and retention time information or MS/MS spectra.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The present disclosure will now be described in greater detail with reference to the drawings in which:

FIG. 1 shows a schematic representation of the use of the compounds of Formula I as reagents for (A) binary and (B) multiplex quantification of one of more carboxylic acids according to embodiments of the present disclosure.

FIG. 2 shows a schematic representation of the use of the compounds of Formula I as reagents for multiplex quantification of one of more carboxylic acids using four reagents having four different isotope mass codings according to one embodiment of the present disclosure.

FIG. 3A shows an ion chromatogram produced by LC-MS analysis of a mixture of 6 acid standards.

FIG. 3B shows one example of a mass spectrum generated for the DmPA labeled acids in one embodiment of the present disclosure.

FIG. 4A shows the ion chromatogram of a human urine sample after differentential isotope labeling with ¹²C₂/¹³C₂-DmPA.

FIG. 4B shows and expanded spectrum obtained at the retention time of about 6.89 min in the ion chromatogram of FIG. 4A.

FIG. 4C shows another mass spectrum where a monoacidic metabolite was detected with a characteristic mass difference of 2 Da from the two main peaks. The

### DETAILED DESCRIPTION OF THE DISCLOSURE

### Definitions

The term “alkyl” as used herein refers to straight and branched chain alkyl groups having 1, 2, 3, 4, 5 or 6 carbon atoms.

The term “salt” means an acid addition salt or basic addition salt.

The term “acid addition salt” as used herein means any organic or inorganic salt of any base compound of the disclosure, or any of its intermediates. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, oxalic acid cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compounds of the invention are more soluble in water and various hydrophilic organic solvents, and generally demonstrate higher melting points in comparison to their free base forms. The selection of the appropriate salt will be known to one skilled in the art.

The term “basic addition salt” as used herein means any organic or inorganic base addition salt of any acid compound of the disclosure, or any of its intermediates. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound or a salt of a compound, wherein molecules of a suitable solvent are incorporated in the crystal lattice. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

“Stable isotopes” of elements as used herein means an isotope of an element having identical numbers of protons and electrons, but having an additional neutron, which increases the molecular weight of the element by one mass unit.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Compounds of the Disclosure

The present disclosure includes a compound of Formula I:

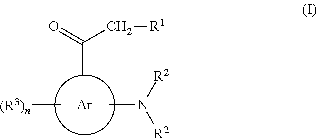

wherein
Ar is phenyl or naphthyl;
R¹ is a suitable leaving group;
R² is C₁₋₁₀alkyl in which one, two or three of the carbon atoms, with the exception of the carbon atom attached to the nitrogen, is optionally replaced with 0 and/or NR⁴ and one or more of the carbon atoms in R² is present as a carbon-13 isotope in amounts greater than the natural abundance of carbon-13 and/or one or more of the oxygen atoms, if present, in R² is present as an oxygen-18 isotope in amounts greater than the natural abundance of oxygen-18;
R³ is selected from C₁₋₆alkoxy and N(C₁₋₆alkyl)₂;
R⁴ is selected from H and C₁₋₆alkyl; and
n is 0, 1, 2, 3 or 4, and salts and solvates thereof.

In an embodiment of the disclosure Ar is phenyl.

In an embodiment of the disclosure, R¹ is a halogen selected from Br, I, Cl and F or R¹ is OTosyl. In a further embodiment R¹ is Br.

In a further embodiment R² is ¹³CH₃, ¹³CH₃CH₂, CH₃¹³CH₂, ¹³CH₃¹³CH₂, (CH₃)₂¹³CH, (¹³CH₃)₂CH, (¹³CH₃)₂¹³CH, ¹³CH₃OCH₂CH₂, CH₃O¹³CH₂¹³CH₂, ¹³CH₃O¹³CH₂¹³CH₂, CH₃¹⁸OCH₂CH₂, or CH₃¹⁸O¹³CH₂¹³CH₂. In another embodiment, R² is ¹³CH₃.

In another embodiment, R³ is CH₃O or CH₃CH₂O and n is 1 or 2. In a further embodiment n is 0.

In another embodiment, R⁴ is H, CH₃ or CH₂CH₃. In a further embodiment R⁴ is H or CH₃.

In another embodiment of the disclosure, when Ar is phenyl, the group N(R²)₂ is attached to the phenyl ring at the position that is para to the C(O)CH₂R¹ group. In another embodiment, when Ar is naphthyl, the group N(R²)₂ is attached to the naphthyl ring at the position that is para to the C(O)CH₂R¹ group.

An exemplary preparation of the compounds of Formula I is shown in Scheme 1. Generally, aminoacetophenones of Formula II, wherein Ar, R³ and n are as defined in Formula I (which are commercially available or are prepared using methods known in the art), are alkylated, for example with a reagent of Formula III, wherein R² is as defined in Formula I (available from Sigma Aldrich), in the presence of a base under conditions to form a compound of the Formula IV, wherein Ar, R², R³ and n are as defined in Formula I. In an embodiment of the disclosure, the alkylation is performed in two steps, with the second alkylation being performed in the presence of a stronger base. In an embodiment, the compound of the Formula IV is reacted with bromine in the presence of an acid, under conditions to prepare the compound of the Formula V, wherein Ar, R², R³ and n are as defined in Formula I, which is then mono-debrominated, for example using diethyl phosphite in the presence of a base, under conditions to form the compound of the Formula I, wherein R¹ is Br and Ar, R², R³ and n are is as defined in Formula I. The compounds of Formula I, wherein R¹ is Br are converted to other compounds of Formula I, wherein R¹ is an alternative suitable leaving group, such as I and Otosyl, using methods known in the art.

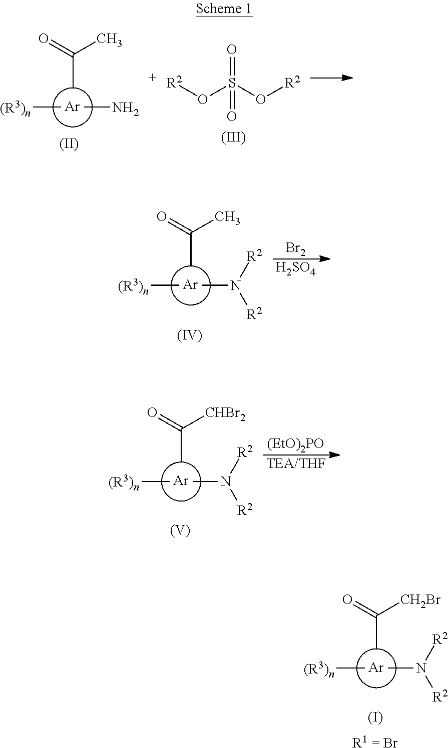

The present application also includes the carboxylic acids derivatized with a compound of Formula I as defined above. It will be appreciated by those skilled in the art that any compound comprising at least one carboxylic acid moiety is capable of reacting with the compounds of Formula I to form the corresponding ester. It is an embodiment of the disclosure that the carboxylic acid is a metabolite found in a biological sample or an aqueous sample such as an agricultural or environmental sample. In a further embodiment of the disclosure, the biological sample is blood, plasma, serum or urine.

Accordingly, also included in the present disclosure in a method of preparing an ester of a carboxylic acid comprising reacting a compound comprising at least one carboxylic acid with a compound of Formula I as defined above in the presence of a suitable base under conditions to form the ester of the one or more carboxylic acids.

The formation of the esters VI, wherein Ar, R², R³ and n are as defined in Formula I and R is any residue of a carboxylic acid, using the compound of Formula I is shown generally in Scheme 2:

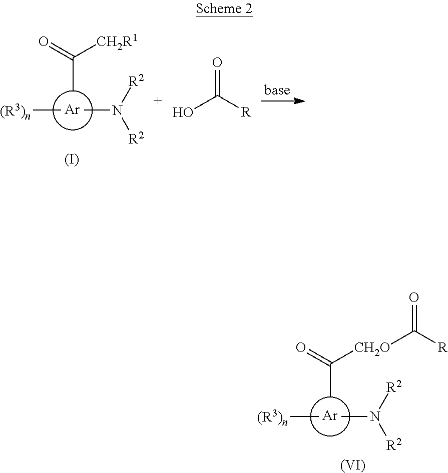

In an embodiment of the disclosure, the base is a non-nucleophilic organic amine base, for example a trialkylamine, such as triethylamine. In a further embodiment the formation of the compounds of Formula VI is performed in a suitable reaction solvent, such as a buffer at a pH of about 7 to about 10, at a temperature of about 60° C. to about 130° C., suitably about 80° C. to about 95° C., for about 5 minutes to about 60 minutes, suitably about 10 minutes to about 30 minutes.

In a further embodiment, the present disclosure also includes a library comprising, consisting essentially of or consisting of two or more compounds of Formula VI as defined above wherein each of the two or more compounds of Formula VI contains a different residue “R” corresponding to a known carboxylic acid. In a further embodiment, the amount of each compound of Formula VI is known so that the library represents a standard or control sample that is used to quantitatively determine an amount of one or more of the carboxylic acids in a test sample.

The natural abundance of various isotopes in nature has been approximated, for example, in the CRC Handbook of Chemistry and Physics, (D. R. Lide, Ed. 89^{th} Edition, 2008-2009, CRC Press Inc. U.S.). The most abundantly occurring form of carbon, the carbon-12 (¹²C) isotope, is approximately 98.90% abundant in nature. The stable carbon-13 (¹³C) isotope, by contrast, is only approximately 1.10% naturally abundant. The most abundantly occurring form of oxygen, the carbon-16 (¹⁶O) isotope, is approximately 99.765% abundant in nature. The stable oxygen-18 (¹⁸O) isotope, by contrast, is only approximately 0.1995% naturally abundant. Accordingly, standard molecules known in the art will generally have incorporated therein various isotopes in these respective percentages of natural abundance. The present disclosure, however, relates to analogs of standard compounds, for example, compounds of Formula I and VI, in which the less naturally abundant stable isotope is selectively incorporated into the structure at desired positions thereof, such that a given analog will have a characteristic molecular weight different from the molecular weight of its corresponding standard compound.

Isotopically labeled carboxylic acid esters, for example the compounds of Formula VI, according to the present disclosure suitably differ from their corresponding standard carboxylic acid ester by a molecular weight of between 2 and 16 atomic mass units (amu's). In particular, it is desirable that isotopes be incorporated in such a manner, and the mass difference be sufficient such that, the mass spectrometric molecular ion peaks of the isotopically-labeled derivative and standard carboxylic acid are distinguishable.

A benefit offered by all isotopically labeled analog internal standards reported herein is that their chemical properties are essentially identical to the target analyte. This means that during sample extraction and workup there can be no or very little differential loss of internal standard versus the target analyte due to differing chemical properties, as may be the case with a chemical analog with differing chemical properties. Again, this translates to an inherently more accurate analytical method when using the compounds of Formula I as defined herein as isotope mass-coded derivatives.

### Methods of the Disclosure

The compounds of Formula I as defined herein are useful for quantitative analysis of carboxylic acids in samples, for example, biological samples. In an embodiment, the quantitative analysis is performed using differential isotope labeling methods. In general, this method involves reacting a first sample comprising one or more carboxylic acids with a compound of Formula I as defined above. A second comparative (or standard) sample comprising one or more carboxylic acids is reacted with a compound having the same structure as that of the compound of Formula I, but that includes an amount of carbon-13 and oxygen-18, if present, that corresponds to its natural abundance. The first and second reacted samples are then analyzed. In one embodiment, the first and second reacted samples are combined and then analyzed by mass spectrometry. The mass spectral analysis of the first and second reacted samples provides quantitative information relating to the amount of carboxylic acids in the first and second samples. This is done by analyzing the peak intensity ratio of the isotope-labeled samples and can be done as a relative quantification of the carboxylic acids in two comparative samples or absolute quantification of the carboxylic acids in a sample if the other sample is a standard compound with known concentration.

Accordingly, the present disclosure also includes a method of quantifying one or more carboxylic acids:
(a) reacting an aliquot of a first sample with a compound of Formula I as defined herein in the presence of a suitable base under conditions to form a first reaction mixture comprising a first ester of the one or more carboxylic acids;
(b) reacting an aliquot of a second sample with a compound having the same structure as the compound of Formula I, with the exception that R² contains an amount of carbon-13 and oxygen-18, if present that corresponds to their natural abundance, in the presence of a suitable base under conditions to form a second reaction mixture comprising a second ester of the one or more carboxylic acids;
(c) combining the first and second reaction mixtures; and
(d) subjecting the combination of (c) to mass spectrometry analysis and quantifying an amount of carboxylic acids in the first sample relative to the second sample.

In an embodiment of the disclosure, one of the first or second sample comprises one or more standard carboxylic acids with known concentrations and the method provides an absolute quantification of the one or more carboxylic acids in the other of the first or second sample. In a further embodiment, the first and second samples are comparative samples, such as urine, plasma, serum or blood samples, from diseased and healthy individuals.

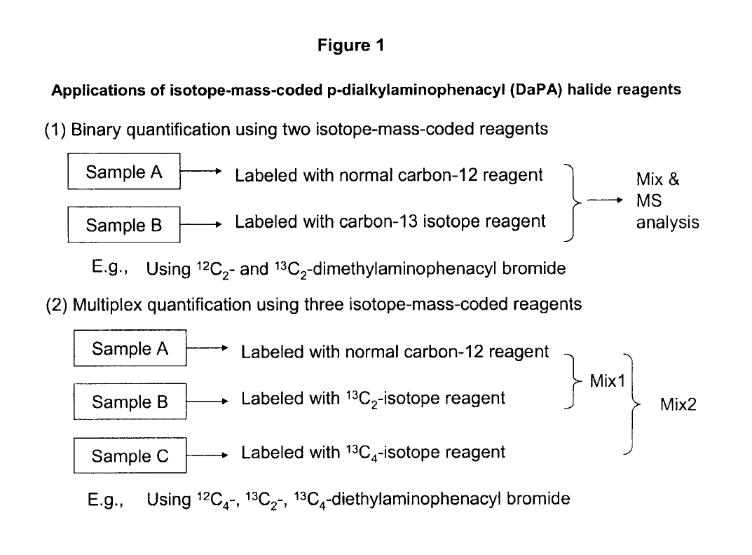

FIG. 1A shows a schematic representation one embodiment of the use of the compounds of Formula I as defined herein as reagents for binary quantification of one of more carboxylic acids.

The present disclosure also includes a multiplex method of quantifying one or more carboxylic acids in three or more samples comprising:
(a) reacting aliquots of two or more samples, separately, each with a different compound of Formula I as defined herein having a different isotope mass, in the presence of a suitable base under conditions to form two or more reaction mixtures each comprising a different ester of the one or more carboxylic acids;
(b) reacting an aliquot of a standard sample with a compound having the same structure as the compound of Formula I, with the exception that R² contains an amount of carbon-13 and oxygen-18, if present, that corresponds to their natural abundance, in the presence of a suitable base under conditions to form a standard reaction mixture comprising a standard ester of the one or more carboxylic acids;
(c) combining, in any order and combination, the reaction mixtures of (a) and (b); and
(d) subjecting the combination of (c) to mass spectrometry analysis and quantifying an amount of carboxylic acids in the two or more samples relative to the standard sample.

In an embodiment of the disclosure, where the standard sample comprises one or more standard carboxylic acids with known concentrations, the method provides an absolute quantification of the one or more carboxylic acids in the two or more samples.

In an embodiment of the disclosure, in the multiplex method, the two or more samples are samples from diseased individuals and the standard sample is as sample from healthy individuals. In another embodiment, the sample is from urine, plasma, serum or blood.

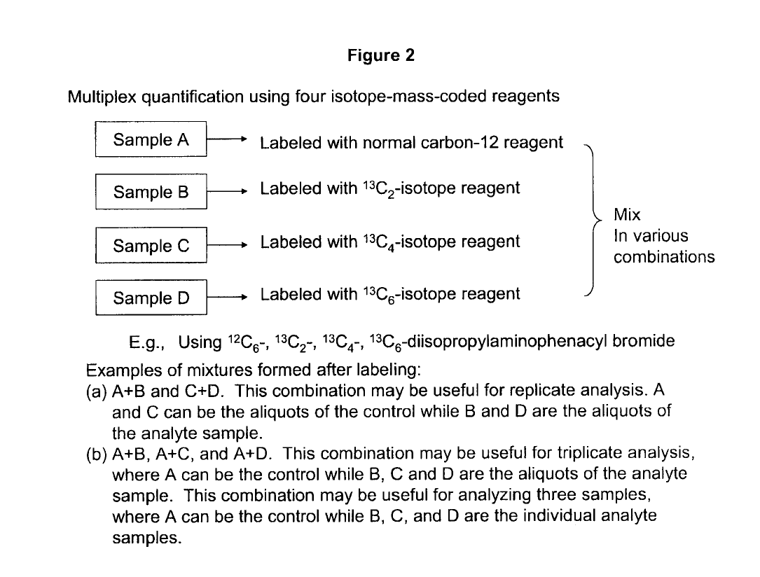

FIGS. 1B and 2 shows schematic representations of one embodiment of the use of the compounds of Formula I as defined herein as reagents for multiplex quantification of one of more carboxylic acids.

It is an embodiment of the binary and multiplex methods of the present disclosure that the mass spectrometry analysis is liquid chromatography/mass spectrometry (LC/MS), flow injection mass spectrometry, or direct sample introduction mass spectrometry.

In an embodiment of the disclosure, the LC comprises the use of reversed phase liquid chromatography, although a person skilled in the art would understand that the specific form of LC will vary depending on the identity of the carboxylic acids(s). In a further embodiment the mass spectrometry (MS) comprises the use of electrospray ionization (ESI) mass spectrometry.

The stable isotope-labeled compounds of the present disclosure are demonstrably useful for improving the efficiency of methodologies for analysis of biological samples for the presence of carboxylic acids and for determining the concentrations of carboxylic acids. In particular, the carbon-13 and/or oxygen-18 labeled compounds of Formula I of the present disclosure are especially useful compounds in the analysis of carboxylic acids in samples, particularly biological samples, for example, for metabolite analysis, for metabolome analysis, in pharmacokinetic and pharmacodynamic studies or for quantitative proteomics.

One major targeted area of application of the present disclosure is for metabolomics which involves a large scale analysis of metabolome (all metabolites) in biological samples. In particular the present disclosure is directed to the use of compounds of Formula I as defined herein in generating quantitative information on metabolome changes in comparative samples, such as urine, plasma, serum or blood samples, from diseased and healthy individuals. In this embodiment, a chemical reaction is used to introduce an isotope tag to an analyte(s) in one sample and another mass-different isotope tag is introduced via a separate reaction to the same analyte(s) in a comparative sample (or standard), followed by mixing the two labeled samples for mass spectrometric analysis. The peak intensity ratio of the isotope labeled analyte pair provides the basis of relative quantification of the analyte(s) in the two comparative samples or absolute quantification of the analyte(s) in a sample if the one sample is a standard with a known concentration of analyte(s).

### EXAMPLES

### Methods

A Bruker apex-Qe™ 9.4-T FT-ICR-MS was employed. A Waters Acquity™ BEH C18 column (2.1×50 mm, 1.7 m) was used for fast reverse phase (RP) separation.

### Results

### Example 1

### Preparation of DmPA

Synthesis of ¹³C₂-DmPA was based on a three-step procedure as shown in Scheme 3. The first reaction involved a dimethylation reaction using conditions as described in J. Physical Org. Chem. 1996, 9, 35-40 and J. Org. Chem. 1968, 33, 318-322. The second and third reactions involved bromination and debromination reactions using conditions as described in Tetrahedron Lett. 1998, 39, 4987-4990. The three-step procedure was optimized for the preparation of ˜1 gram of labeling reagent which ensured a good supply of this reagent. In most LC/MS work, only ˜100 μg of the labeling reagent are needed for one sample. Two semi-preparative reversed-phase (RP) separations and normal phase flash chromatography were used to produce high purity reagents. The purity of the labeling reagent was >99.5% by HPLC, UV, MS and NMR analysis.

The synthesis of other isotope reagents, such as p-diethylaminophenacyl bromide with varying numbers of carbon-13 (¹³Cₙ-DePA) and p-diisopropylaminophenacyl bromide containing varying numbers of carbon-13 (¹³Cₙ-DipPA) can be readily performed using a reaction scheme similar to that shown in Scheme 3. In these cases, dimethyl sulfate-¹³C₂ is replaced with diethyl sulfate-¹³C₂ and diisopropyl sulfate-¹³C₂, respectively. It is noted that dimethyl sulfate-¹³C₂ is commercially available, for example from Sigma Aldrich, however diethyl sulfate-¹³C₂ and diisopropyl sulfate-¹³C₂ is made by reacting ethanol or isopropanol with sulfuric acid or SO₂Cl₂ as described in J. Amer. Chem. Soc. 1924, 46, 999 and Compt. Rend. 1929, 188, 261. Isotope-containing ethanol is available, for example from Sigma Aldrich, in various forms, such as ¹²CH₃CH₂OH or ¹³CH₃¹³CH₂OH. Isotope-containing isopropanol is also available, for example from Sigma Aldrich, in forms such as isopropanol-2-¹³C, isopropanol-1,2-¹³C₂ and isopropanol-¹³C₃. Scheme 3 only illustrates one route of preparing DmPA. Alternative routes can also be used to prepare DmPA, DePA or DipPA.

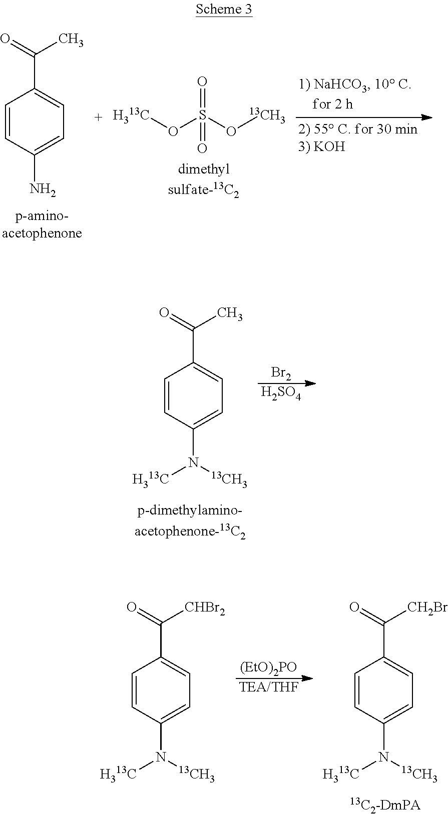

### Example 2

### Labeling of Carboxylic Acids

Scheme 4 shows the labeling reaction using ¹³C₂-DmPA and a carboxylic acid. The labeling procedure was fast (˜15-20 min at 85-90° C. in a water bath), simple and robust. Triethylamine (TEA) was used in Scheme 4 as base. Other examples of bases include triethanolamine (TEOA) and N-methyldiethanolamine.

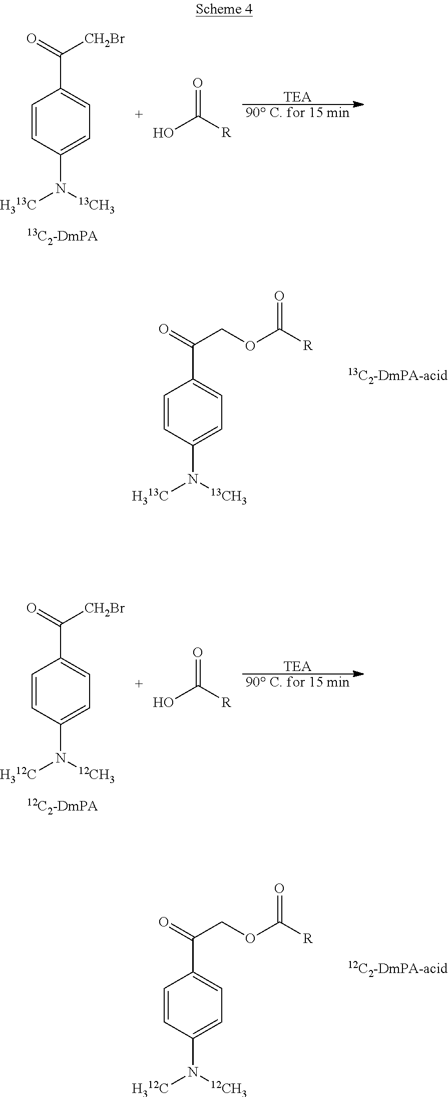

### Example 3

### LC-MS Analysis of Standard Carboxylic Acid Mixtures

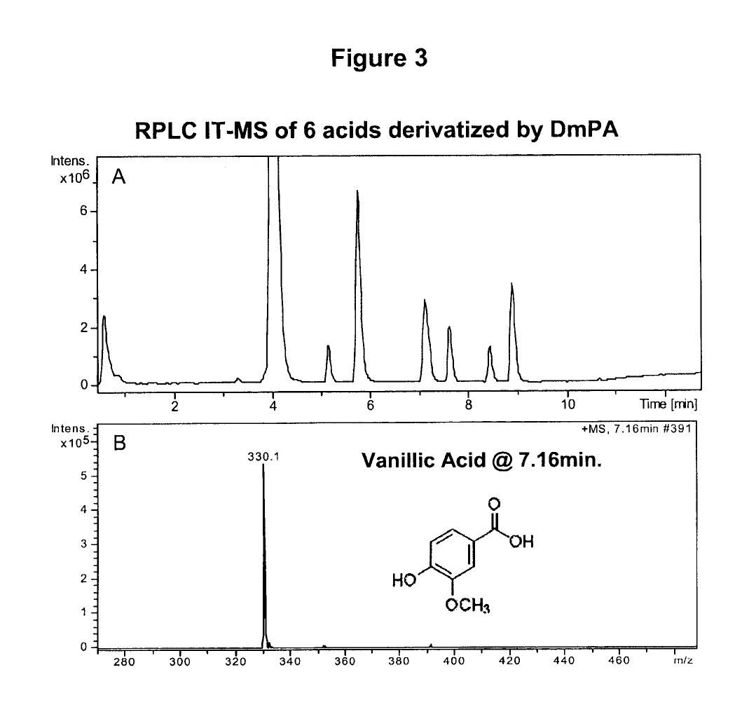

FIG. 3A shows an ion chromatogram produced by LC-MS analysis of a mixture of 6 acid standards. FIG. 3B shows one example of a mass spectrum generated for the DmPA labeled acids. The peak at m/z 330 is from DmPA-vanillic acid.

### Example 4

### Differential Isotope Labeling of Human Urine Sample

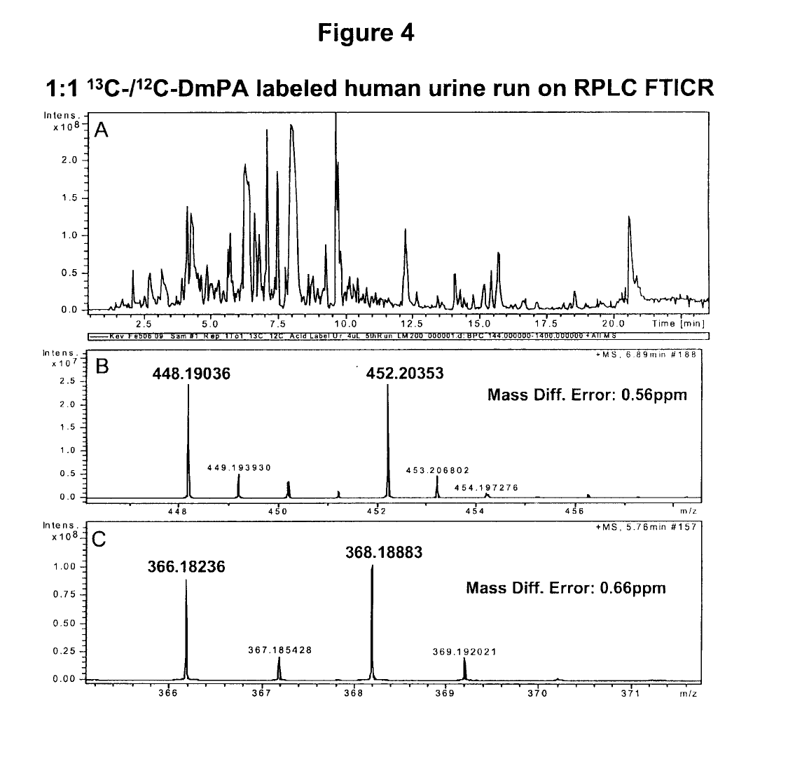

FIG. 4A shows the ion chromatogram of a human urine sample after differentential isotope labeling with ¹²C₂/¹³C₂-DmPA. In this case, the urine sample was divided into two equal aliquots, followed by labeling one aliquot with ¹²C₂-DmPA and another one with ¹³C₂-DmPA. The two labeled aliquots were then mixed and the mixture injected into an LC-MS for analysis. Identification of the peak pair was easy as the light chain and heavy chain labeled metabolites had a mass difference of 2 Da for one DmPA tag attached or 4 Da for two DmPA tags. FIG. 4B shows and expanded spectrum obtained at the retention time of about 6.89 min. The two main peaks separated by 4 Da are from the metabolite containing two carboxylic acid groups (i.e. labeled with two molecules of DmPA or two tags). FIG. 4C shows another mass spectrum where a monoacidic metabolite was detected with a characteristic mass difference of 2 Da from the two main peaks. The peak ratios are used to calculate the relative abundance difference of the metabolite in two comparative examples. As expected, the light chain and heavy chain labeled metabolites in this case had an intensity ratio of close to 1.

### Example 5

### Relative Metabolome Quantification

Relative quantification of carboxylic acids in two comparative metabolome samples can be done by making ¹²C-DmPA derivatives from one sample and ¹³C-DmPA derivatives from the other sample, followed by mixing the two labeled samples and injecting the mixture into LC/MS for analysis. The intensities of the mass spectral peak pairs are compared to generate information on the relative quantity differences of the metabolites in the two samples.

### Example 6

### Absolute Metabolome Quantification

With the availability of a DmPA compound library, it is possible to determine the absolute concentration of each metabolite in a biological sample, as long as the carboxylic acid analyte standard is present in the library. A strategy of measuring absolute metabolite concentrations of individual samples is explored using a pooled sample as an internal standard. A pooled sample is prepared by taking aliquots from individual samples and then combining them to form a composite sample. This sample is labeled with ¹²C-DmPA. The ¹³C-DmPA metabolite standards are spiked to an aliquot of the pooled sample, followed by running the mixture in LC/MS. The metabolites present in the pooled sample can be identified based on the retention time match and accurate molecular mass measurement. The metabolite concentration can be determined based on the measured peak abundance ratios of ¹³C-/¹²C-DmPA derivatives and the amount of the ¹³C-standards spiked to the sample. To determine the concentrations of metabolites in the individual samples, each sample is labeled by ¹³C-DmPA and then mixed with an aliquot of the ¹²C-DmPA pooled sample. Based on individual metabolites already measured in the pooled sample, the absolute concentration of each metabolite in the individual samples can be determined.

To demonstrate the utility of this method of using a pooled sample as an internal standard, human urine samples are collected over five consecutive mornings from the same healthy individual. A pooled urine sample is then prepared by mixing equal volume aliquots of “Day-1” to “Day-5” urine samples. The carboxylic acid-¹²C-DmPA standards are grouped into mixtures to minimize the complexity of the samples and reduce the possibility of ion suppression in LC/MS (i.e., the spiked standards may suppress the analyte signals in the urine sample). Note that, depending on the type of biological samples analyzed, the concentrations of individual standards in the mixture may be adjusted so that the ¹³C-/¹²C-DmPA peaks do not fall off the linear dynamic range of relative quantification. Each group of mixture is esterified with ¹³C-DmPA and then spiked into the ¹²C-DmPA pooled urine for absolute quantification.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

(1) Casetta, B.; Daniels, S.; Stantick, W.; Cox, D.; Nimkar, S.; Cardenas, J.;
Gamble, T. Clin. Biochem, 2006, 39, 1099-1099.
(2) Fukusaki, E.; Harada, k.; Bamba, T.; Kobayashi, A. J Biosci Bioeng, 2005. 99, 75-77
(3) Yang, W,; Regnier, F.; Sliva, D.; Adamec, J, JChromatogr, B 2008, 870, 223-240.
(4) Shortreed, M. R.; Lamos S. M.; Frey, B. L.; Philips, M. F.; Patel, M.; Belshaw, P. J,; Smith, L. M. Anal. Chem. 2006, 78, 6398-6403.
(5) Guo, K,; Ji, C,; Li, L,; Anal Chem 2007, 79, 8631-8638.
(6) Ji, C,; W. L,; Ren, X. D.; El-Kattan, A. F.; Kozak, R.; Fountain, S.; Lepsy, C. Anal. Chem. 2008, 80, 9195-9203.
(7) Abello, N.; Geurik, P.; van der Toorn, M.; van Oosterhout, A.; Lugtenburg, J.; van der Marel, G.; Kerstjens, H.; Postma, D.; Overkleeft, H.; Bischoff, R. Anal Chem. 2008, 80, 9171-9180.
(8) O'Maille, G.; Go, E.; Hoang, L.; Want, E.; Smith, C.; O'Mallie, P.; Nordstrom, A,; Morita, H.; Qin, C.; Uritboonthai, W.; Apon, J.; Moore, R.; Garrett, J.; Siuzdak, G.; Spectroscopy 2008, 22, 327-343.
(9) Huang, X.; REgnier, F. Anal Chem 2008, 80, 107-114.
(10) Mashego, M.; Wu, L,; Van Dam, J.; Ras, C.; Vinke, J.; Van Winden, W.; Van Gulik, W.; Heijnen, J. Biotechnoi Bioeng, 2004, 85, 620-628.
(11) Wu, L,; Mashego, M.; van Dam, J,; Proell, A.; Vinke, J.; Ras, C.; van Winden, W,; van Gulik, W.; Heijnen, J. Anal Biochem 20058, 336, 164-171.
(12) Kim, J.; Harada, K.; Bamba, T.; Fukusaki, E.; Kobaysahi, A.; Biosci Biotechnol Biochem 2005, 69, 1331-1340.
(13) Hegman, A., Schulte, C.; Cui, Q.; Lewis, I.; Huttlin, E.; Eghbalnia, H.; Harms, A.; Ulrick, E.; Markley, J.; Sussman, M. Anal Chem 2007, 79, 6912-6921.
(14) Metz, T.; Zhang, Q.; Page, J.; Shen, Y.; Callister, S.; Jacobs, J.; Smith, R.; Biomark Med 2007, 1, 159-185.
(15) Bennett, B., Yuan, J.; Kimball, E.; Rabinowitz, J. Nat Protoc 2008, 3, 1299-1311.
(16) Madalinski, G.; Godat, E.; Alves, S.; Lesage, D.; Genin, E.; Levi, P.; Labarre, J.; Tabet J.; Ezan, E.; Junot, C. Anal Chem 2008, 80, 3291-3303.
(17) Guo, K.; Li, L; Anal Chem 2009, 81, 3919-3932.
(18) Eisenbert, Eugene J.; Cundy, Kenneth C. High performance liquid chromatographic determination of cytosine-containing compounds by precolumn fluorescence derivatization with phenacyl bromide: application to antivirial nucleosides and nucleotides. Journal of Chromatography, B; Biomedical Application 1996, 679, 119-127.
(19) Hanis, Tomas; Smrz Miroslav; Klir, Pavel; Macek, Karel; Klima, Josef; Base, Jiri; Deyl, Zdenek. Determination of fatty acids as phenacyl esters in rat adipose tissues and blood vessel walls by high-performance liquid chromatography. Journal of Chromatography 1988, 452, 443-57.
(20) Reid, Gavin E.; Roberts, Kade d.; Simpson, Richard J.; O'Hair, Richard A. J. Journal of the American Society of Mass Spectrometry 2005, 16, 1131-1150.
(21) Amunugama, Mahasilu; Roberts, Kade D.; Reid, Gavin E. Journal of the American Society for Mass Spectrometry 2006, 17, 1631-1642.

1. A compound of Formula I: 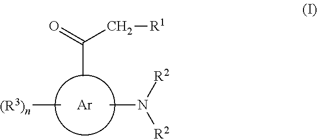
wherein
Ar is phenyl or naphthyl;
R¹ is a suitable leaving group;
R² is C₁₋₁₀alkyl in which one, two or three of the carbon atoms, with the exception of the carbon atom attached to the nitrogen, is optionally replaced with O and/or NR⁴ and one or more of the carbon atoms in R² is present as a carbon-13 isotope in amounts greater than the natural abundance of carbon-13 and/or one or more of the oxygen atoms, if present, in R² is present as an oxygen-18 isotope in amounts greater than the natural abundance of oxygen-18;
R³ is selected from C₁₋₁₀alkoxy and N(C₁₋₆alkyl)₂;
R⁴ is selected from H and C₁₋₆alkyl; and
n is 0, 1, 2, 3 or 4, and
salts and solvates thereof.

2. The compound of claim 1, wherein R¹ is a halogen selected from Br, I, Cl and F or R¹ is OTosyl.

3. (canceled)

4. The compound of claim 1, wherein R² is ¹³CH₃, ¹³CH₃CH₂, CH₃¹³CH₂, ¹³CH₃¹³CH₂, (CH₃)₂¹³CH, (¹³CH₃)₂CH, (¹³CH₃)₂¹³CH, CH₃O¹³CH₂¹³CH₂, ¹³CH₃O¹³CH₂¹³CH₂, CH₃¹⁸OCH₂CH₂, or CH₃¹⁸O¹³CH₂¹³CH₂.

5. (canceled)

6. The compound of claim 1, wherein R³ is OCH₃ or OCH₂CH₃ and n is 1 or 2.

7. The compound of claim 1, wherein n is 0.

8. (canceled)

9. The compound of claim 1, wherein Ar is phenyl.

10. The compound of claim 9, wherein N(R²)₂ is attached to the phenyl ring at the position that is para to the C(O)CH₂R¹ group.

11. A method of preparing an ester of a carboxylic acid comprising reacting a compound comprising at least one carboxylic acid with the compound of Formula I as defined in claim 1 in the presence of a suitable base under conditions to form the ester of the one or more carboxylic acids.

12. The method of claim 11, wherein the carboxylic acid is a metabolite found in a biological sample.

13. The method of claim 12, wherein the biological sample is blood, plasma, serum or urine.

14. A method of quantifying one or more carboxylic acids:
(a) reacting an aliquot of a first sample with a compound of Formula I as defined in claim 1 in the presence of a suitable base under conditions to form a first reaction mixture comprising a first ester of the one or more carboxylic acids;
(b) reacting an aliquot of a second sample with a compound having the same structure as the compound of Formula I, with the exception that R² contains an amount of carbon-13 and oxygen-18, if present, that corresponds to their natural abundance, in the presence of a suitable base under conditions to form a second reaction mixture comprising a second ester of the one or more carboxylic acids;
(c) combining the first and second reaction mixtures; and
(d) subjecting the combination of (c) to mass spectometry analysis and quantifying an amount of carboxylic acids in the first sample relative to the second sample.

15. The method of claim 14, wherein one of the first or second sample comprises one or more standard carboxylic acids with known concentrations and the method provides an absolute quantification of the one or more carboxylic acids in the other of the first or second sample.

16. The method according to claim 15, wherein the first and second samples are comparative samples, such as urine, plasma, serum or blood samples, from diseased and healthy individuals.

17. A multiplex method of quantifying one or more carboxylic acids in three or more samples comprising:
(a) reacting aliquots of two or more samples, separately, each with a different compound of Formula I as defined in claim 1 having a different isotope mass, in the presence of a suitable base under conditions to form two or more reaction mixtures each comprising a different ester of the one or more carboxylic acids;
(b) reacting an aliquot of a standard sample with a compound having the same structure as the compound of Formula I, with the exception that R² contains an amount of carbon-13 and oxygen-18, if present, that corresponds to their natural abundance, in the presence of a suitable base under conditions to form a standard reaction mixture comprising a standard ester of the one or more carboxylic acids;
(c) combining, in any order and combination, the reaction mixtures of (a) and (b); and
(d) subjecting the combination of (c) to mass spectrometry analysis and quantifying an amount of carboxylic acids in the two or more samples relative to the standard sample.

18. The method of claim 17, wherein the standard sample comprises one or more standard carboxylic acids with known concentrations and the method provides an absolute quantification of the one or more carboxylic acids in the two or more samples.

19. The method of claim 18, wherein the two or more samples are samples from diseased individuals and the standard sample is as sample from healthy individuals.

20. The method of claim 14, wherein the mass spectrometry analysis is liquid chromatography/mass spectrometry, flow injection mass spectrometry, or direct sample introduction mass spectrometry.

21. A use of a compound of Formula I as defined in claim 1 for generating quantitative information on metabolome changes in comparative samples, such as urine, plasma, serum or blood samples, from diseased and healthy individuals.

22. A library comprising, consisting essentially of or consisting of two or more esters of a carboxylic acid, wherein the esters are formed by reaction of a compound of Formula I as defined in claim 1 with the carboxylic acid in the presence of a base.

23. The library according to claim 22, wherein each of the two or more esters of a carboxylic acid is present in a known amount so that the library represents a standard or control sample that is used to quantitatively determine an amount of two or more of the carboxylic acids in a test sample.
